(19) United States
(12) Patent Application Publication
(10) Pub. No.: US 2011/0135726 A1
(43) Pub. Date: Jun. 9, 2011

(54) HYDROGEL COMPOSITES AND WOUND DRESSINGS

(75) Inventors: Hugh Semple Munro, Warwickshire (GB); Philip Andrews, Marlborough Wiltshire (GB); Charles Jeffrey, Marlborough Wiltshire (GB)

(73) Assignee: FIRST WATER LIMITED, Marlborough Wiltshire (GB)

(21) Appl. No.: 12/866,611

(22) PCT Filed: Feb. 6, 2009
(86) PCT No.: PCT/GB2009/050121

§ 371 Date: Jan. 11, 2011

Related U.S. Application Data
(60) Provisional application No. 61/026,920, filed on Feb. 7, 2008.

(30) Foreign Application Priority Data
Feb. 7, 2008 (GB) 0802284.0

(51) Int. Cl. A61K 9/14; A61P 17/02

(52) U.S. Cl. 424/484

(57) ABSTRACT

The present invention provides an absorbent hydrogel composite for use in the manufacture of an article for application to a fluid-emitting surface, e.g. a wound, the composite having a laminar structure comprising first and second layers, the first layer being a surface contacting layer comprising a porous net structure having a surface contacting face and an outwardly directed face, the second layer comprising a low-crosslinked absorbent hydrogel disposed over the outwardly directed face of the first layer and arranged so that in use it is in fluid flow communication with the surface through apertures of the net structure.

### FIELD OF THE INVENTION

The present invention relates to absorbent hydrogel composites and wound dressings containing them, and more particularly to sheet hydrogel composites suitable for use in wound (including burn) dressings and other applications where absorption of fluid is required. The invention also relates to processes for the manufacture of the novel hydrogel composites, and to uses of the compositions.

The expressions “hydrogel” and “hydrogel composites” used herein are not to be considered as limited to gels which contain water, but extend generally to all hydrophilic gels and gel composites, including those containing organic non-polymeric components in the absence of water.

### BACKGROUND OF THE INVENTION

The use of hydrogels in wound dressings has been described in, for example, U.S. Pat. No. 5,204,110 issued to Cartmel, U.S. Pat. No. 4,979,946 issued to Gilman, U.S. Pat. No. 4,746,514 issued to Warne and U.S. Pat. No. 5,695,777 issued to Donovan. The disclosure of these documents is incorporated herein by reference.

In U.S. Pat. No. 5,695,777, Donovan teaches that hydrogels left in direct contact with the wound can cause maceration and describes an absorptive hydrogel dressing in which the hydrogel does not directly contact the wound. The isolation of the hydrogel was achieved by the use of a “wound contacting layer” comprising a porous medical grade polymer in direct contact with the wound, “wicking means” being also provided, so that the structure is capable of wicking or otherwise transferring wound fluid away from the wound to the hydrogel. The hydrogels described were based on the polymerisation of 2-acrylamido-2-methylpropane sulphonic acid or its salts. No information was disclosed as to any potential therapeutic properties, for example protease sequestration or inhibition of inflammation, the complement and/or kinin cascade, associated with the hydrogel directly. The use of therapeutic agents added to the hydrogel to tailor the dressing to the needs of specific wounds was generally described. The presence of the porous polymer layer results in a dressing with no adhesion characteristics necessitating the use of an adhesive overlayer in order for the dressing to held in place over the wound.

WO-A-2007/007115 (First Water Limited), the disclosure of which is incorporated herein by reference, describes a hydrogel dressing in direct contact with the wound, to provide in use a controlled-moisture environment for the wound and selective uptake of proteins and ions from the wound, to stimulate and/or maintain the wound healing process.

The present invention is based on the realisation that the evidence of WO-A-2007/007115 shows very strong analogies between the hydrogel and certain natural glycosminoglycans of a normal healing wound, and in particular certain sulphonated glycosaminoglycans of the extracellular matrix such as heparin, from which we can now confidently extrapolate the findings to predict activity of the same hydrogels against other conditions which are attenuated by such glycosminoglycans, particularly inflammation and the complement and kinin cascades, and also the blood clotting response. The hydrogel provides a moist wound healing environment where the water levels are controlled to avoid the disadvantages of too much or too little moisture.

The hydrogels described in WO-A-2007/007115 may optionally include within the gel a reinforcing fabric, such as a spun bonded polyester. It has been noticed that these hydrogels, on absorbing large quantities of fluid, swell to a high degree and can be prone to breakdown of the contiguous structure of the sheet. This may then require additional cleansing during dressing changes. The breakdown of the hydrogel structure may also encourage odour release from the wound.

While the prior art wound dressings comprising hydrogels represent a significant development in the area of absorbent hydrogels there is still a need for stronger materials having the desired absorption, wound stimulation and other characteristics offered by hydrogel technology.

### BRIEF DESCRIPTION OF THE INVENTION

The present invention is based on our surprising further finding that relatively strong absorbent hydrogel wound dressings can be made in a convenient manner with little or no breakdown of the hydrogel on fluid uptake whilst maintaining direct contact with the wound.

The minimisation of the break-up of the absorbent hydrogel on uptake of fluid is achieved by utilising hydrogel formulations that incorporate low levels of crosslinking agent.

Such absorbent hydrogels can have a high level of adhesion, which may not be appropriate in all situations and for all wound care treatments. It would be desirable to produce a hydrogel composition that can effectively treat wounds, particularly chronic wounds, and which is more resistant to break-up on uptake of high levels of fluid, with a lower level of adhesion to the skin. By positioning a porous net or mesh structure into the wound contacting surface of the low-crosslinked absorbent hydrogel, it has been surprisingly found that a wound dressing resistant to break up on the absorption of fluid and having acceptable and useful levels of adhesion to mammalian skin can be made.

The combination of the beneficial therapeutic properties of the hydrogel with a low crosslinking level and a surface orientated net structure to control the skin contacting adhesion levels in the wound dressings of the present invention represents a significant advancement in the use of hydrogels dressings for wound healing. An additional benefit of the absorbent hydrogel wound dressing of the present invention is that a release layer may be adhered to the wound contacting surface, which simplifies manufacturing processes for making the dressings from rolls of hydrogel.

According to a first aspect of the present invention, there is provided an absorbent hydrogel composite for use in the manufacture of an article for application to a fluid-emitting surface, e.g. a wound, the composite having a laminar structure comprising first and second layers, the first layer being a surface contacting layer comprising a porous net structure having a surface contacting face and an outwardly directed face, the second layer comprising a low-crosslinked absorbent hydrogel disposed over the outwardly directed face of the first layer and arranged so that in use it is in fluid flow communication with the surface through apertures of the net structure. The arrangement may, for example, be such that in use on a wound the first layer maintains effective structural integrity of the low-cross-linked absorbent hydrogel of the second layer as the dressing absorbs fluid.

The first layer of the composite is preferably embedded into the second layer to such an extent that in use the second layer contacts the surface through the apertures of the net structure.

According to a second aspect of the present invention, there is provided a wound dressing comprising such an absorbent hydrogel composite.

The wound dressing preferably has a laminar structure comprising first and second layers, the first layer being a wound contacting layer comprising a porous net structure having a wound contacting face and an outwardly directed face, the second layer comprising a low-crosslinked absorbent hydrogel disposed over the outwardly directed face of the first layer and in fluid flow communication with the wound through apertures of the net structure.

The first layer of the wound dressing is preferably embedded into the second layer to such an extent that in use the second layer contacts the wound through the apertures of the net structure.

The first layer may, if desired, consist essentially of a porous net structure, and may for example consist of the porous net structure.

The second layer may, if desired, consist essentially of an absorbent hydrogel, and may for example consist of the absorbent hydrogel.

It is most preferred that the arrangement is such that the strongly adhesive properties of many low-cross-linked absorbent hydrogels are controlled and particularly the adhesiveness of the composite is adjusted to the desirable range for the intended use. For example, as used in a wound dressing, or in other applications on the human skin, it is desired that the dressing should be mildly tacky to the skin/wound, so that it is kept in place for the desired period of time, and yet that it can be readily peeled off the skin without undue discomfort and without pulling hairs. In addition, for use in a wound dressing, or in other applications on the human skin, the composite should not irritate the skin during the period of use.

The porous net structure of the first layer may suitably directly contact the absorbent hydrogel of the second layer. In this embodiment, preferably the mesh size of the net is large enough that the absorbent hydrogel of the second layer is exposed by the net apertures and thus comes into direct contact with the wound.

The absorbent hydrogel may optionally may be associated with a reinforcing sheet, for example a reinforcing fabric, which may suitably be embedded in (integral with) the hydrogel.

The absorbent hydrogel may suitably possess intrinsic therapeutic properties, for example as described in WO-A-2007/007115, or may include additives for providing therapeutic or other physiological benefits. Further details of possible additives are provided below.

The second layer may itself be multilamellar, i.e. formed of multiple component layers (“sub-layers”). It is preferred that at least one of such sub-layers will comprise an absorbent hydrogel, provided that there is the required fluid flow communication between the hydrogel and the fluid source through the apertures of the net structure. In such an embodiment, the hydrogel composition of the sub-layer closer or closest to the fluid source may be the same composition as the one or more of the other sub-layer or sub-layers. Alternatively, the hydrogel composition of the sub-layer closer or closest to the wound may be a composition which is different from one or more of the other sub-layer or sub-layers, or any combination thereof.

The absorbent hydrogel layer, or any absorbent hydrogel sub-layer thereof, may, if desired, consist essentially of an absorbent hydrogel material, for example, it may consist of an absorbent hydrogel material.

The second layer may suitably be permeable to oxygen and water vapour, to permit the transpiration of fluid and vapour across the outward face of the composite and dressing, enabling a vapour diffusion flow through the composite and dressing.

The composite and dressing may optionally comprise at least one further (third) layer. The third layer, when present, is suitably in direct contact with the second layer and overlying the outward face of the second layer.

The second layer in this embodiment preferably has oxygen and vapour permeability for the transpiration of at least part of the fluid diffusing through the composite and dressing. The third layer may comprise polymer films and/or foams and/or fibres. The third layer may be continuous or foraminous. The third layer may itself be uni- or multilamellar. The third layer may, for example, comprise polyolefins, polyesters, polyurethanes, carboxymethyl cellulose, hydrocolloids, hydrogels, or any mixture or combination thereof.

Depending on the intrinsic vapour permeability of the second layer, the third layer may be fenestrated, i.e. provided with a central cut-out area (window) over the central portion of the second layer, so that the desired level of transpiration of fluid through the composite or dressing is achieved.

The third layer may be of larger, smaller or the same surface area than the other layers. When the third layer is of a larger surface area, the portion thereof that extends beyond the margin of the other layers may include a peripheral skin contacting adhesive on its underside, enabling adhesive contact with the skin area surrounding a wound.

The third layer may, if desired, consist essentially of a polymer film and/or foam and/or fibres, and may for example consist of polymer film and/or foam and/or fibres.

If the third layer has the same surface area as the first and second layers, the dressing may comprise at least one further (fourth) layer, in contact with the third layer and extending beyond the margins thereof, the portion of the fourth layer extending beyond the margins of the other layers suitably including a peripheral contact adhesive on its underside, enabling adhesive contact with the skin area surrounding a wound. In that embodiment, the fourth layer or different portions thereof may be continuous or foraminous, or any combination thereof. The fourth layer may be fenestrated, as described above for the third layer. For example, the fourth layer may comprise a continuous sheet over its area or may possess a fenestrated or perforated region over the area defined by the other three layers. If the third layer is of smaller surface area than the first and second layers, then the further layer(s) may surround or overlie the third layer. The fourth layer may comprise polymer films and/or foams and/or fibres, continuous or foraminous. The fourth layer may be uni- or multilamellar. The fourth layer may, for example, comprise polyolefins, polyesters, polyurethanes, carboxymethyl cellulose, hydrocolloids, hydrogels, or any mixture or combination thereof.

The fourth layer may, if desired, consist essentially of a polymer film and/or foam and/or fibres, and may for example consist of polymer film and/or foam and/or fibres.

Generally speaking, however the composite or dressing is constructed, it is preferred that fluid, oxygen and vapour can diffuse in controlled manner towards and away from the fluid source in use. This controlled diffusivity of the composite or dressing is particularly desirable in the wound-contacting (central) region of a dressing.

The wound contacting first layer effects transfer of fluid away from a wound whilst enabling the hydrogel (preferably having therapeutic properties) to be in direct contact with the wound. The presence of the surface orientated net structure lowers to a useful level the unacceptable adhesive properties of the absorbent hydrogel in direct contact with the wound and surrounding skin.

The preferred embedding of the net structure in the wound-directed surface of the hydrogel also facilitates the direct contact of the absorbent hydrogel with the wound. If the surface area of the dressing is greater than that of the wound, the presence of the absorbent hydrogel at the surface of the dressing facilitates the placement and adhesion of the dressing over the wound area.

The wound dressing may also comprise therapeutic agents, in the net structure, the absorbent hydrogel and or the third or more layers.

For forming any required component part of the composite sheet, generally conventional preparation methods, or methods described in the prior art acknowledged above, may be used. Curing of a pre-gel can suitably be by polymerisation, although evaporation of a solvent from a liquid pre-gel which comprises an organic solvent solution of a pre-formed polymer—to leave behind a dry residue of the polymer—is not excluded from the ambit of the word “curing” as used herein. Thus, for example, the absorbent layer part of the hydrogel composite for use in the present invention may generally be prepared by a process which comprises polymerising a polymerisable mixture comprising a hydrophilic monomer selected from monomers and monomer mixtures. The polymerisable mixture may include introduced gas bubbles, as described in WO-A-03/077964 and WO-A-2004/052415, the disclosures of which are incorporated herein by reference.

Any polymerisation step in the manufacture of the composite hydrogel sheet or component parts thereof is preferably a free radical polymerisation performed in air using a polymerisation inducing device such as a heat, light (e.g. UV light) or other radiation source which is in relative motion with respect to the polymerisable mixture. In this way, a moving line-wise polymerisation procedure can take place.

The optional third and/or fourth layer is formed of any suitable material, e.g. a polymer (which may be foamed or unfoamed, or any combination thereof) such as polyurethane, or a fabric (which may comprise natural fibres, synthetic fibres or any combination thereof, and may be woven or non-woven). The optional third and/or fourth layer may be in any suitable form or structure, e.g. a web, film, sheet, net or any combination thereof.

The optional third and/or fourth layer may have a skin contacting adhesive, for example a pressure sensitive adhesive, on the underside thereof, to facilitate contact with the area surrounding the wound. The pressure-sensitive adhesive is any suitable skin-compatible adhesive, e.g. a known acrylic-based polymeric pressure-sensitive adhesive; or a bioadhesive hydrogel or gel or a bioadhesive porous plasticised hydrophilic polymer having an internal cellular structure, such as those described in the PCT Patent Applications mentioned above.

In an embodiment, the moisture vapour transmission rate (MVTR) of the absorbent hydrogel composite is from about 150 to about 2500 g/m2/24hours, as measured at 40° C., and 55% relative humidity. MVTR is a known measurement in the field. The method used to measure MVTR in the present application is similar to the method disclosed on page 9 of EP-A-0091800, except that the temperature within the oven for the purposes of the present application is 40° C. and the relative humidity within the oven is 55%. (In EP 0091800, “wet” MVP means WTR). The test fluid used is calcium saline solution, as disclosed in the Examples below. The test for MVTR is further exemplified in the Examples below.

### DETAILED DESCRIPTION OF THE INVENTION

### Construction of the Composite or Wound Dressing

The method of preparation of the hydrogel composites according to the present invention, and of articles (e.g. wound dressings) embodying the same, as well as the uses of the articles, as generally described below, constitute further aspects of the present invention.

The absorbent hydrogel of the wound composite or dressing may comprise one or more layers. The hydrogel of the second layer, which contacts the fluid source, is of sufficiently low crosslinker content to minimise hydrogel break up on the absorption of fluid. The amount of crosslinking agent used is such that when combined with the net structure an acceptable level of adhesion to mammalian skin is achieved (less than 4 N/25 mm in a 180 degree peel test at a speed of 200 mm/minute). Lower levels of crosslinker can further minimise the break-up of the hydrogel, but on combining with the net structure are found to give unacceptable levels of adhesion to the skin.

It has been found that these lower-crosslinked hydrogels can constitute additional layers or sub-layers of the composite that do not directly contact the wound but are in direct contact or fluid flow communication with the wound contacting hydrogel. The wound contacting (first) hydrogel layer is at least 100 μm thick and/or suitably has a weight of at least about 100 g/m². Details of the crosslinker and the amount used are further described below. Such an arrangement is also suitable when two or more absorbent hydrogel layers or sub-layers re used.

The first layer comprises a net structure on the face of the composite which is directed to the fluid source (e.g. the wound).

The composite may be manufactured by prepolymerising the crosslinked hydrogel and placing/laminating the latter down on to the net with light pressure, or by placing/laminating the net onto the polymerised hydrogel with light pressure. To those skilled in the art the performance and automation of such procedures will be well understood.

Alternatively, the net may be placed onto the surface of a liquid pre-gel and then the pre-gel cured in situ on the net.

The preferred method is the lamination of the net to the polymerised hydrogel. Still further, the hydrogel layer (second layer) may be present in the form of a sheet having an associated scrim (woven or non-woven fabric, or a net, which may be the same or different from the net of the first layer). The scrim material may be integral with—i.e. suitably embedded within—one or more of any absorbent hydrogel layers that are present in the composite according to the present invention. Such a scrim material may be formed of a material that is natural in origin, synthetic in origin, or partly natural and partly synthetic. The scrim may suitably be in the form of a net or a woven or non-woven fabric. Preferred scrims include those formed from polyolefins, polyamides, polyacrylates, or polyesters, for example non-wovens, foams or nets. The scrim material may, for example, comprise sodium polyacrylate fibres, such as those commercially available under the tradename Oasis™ from Acordis Technical Absorbents Limited. The scrim is preferably provided by introducing it into a laid down (e.g. cast) layer of a pre-gel liquid precursor for the hydrogel layer, before curing, so that the liquid pre-gel covers and surrounds the scrim. On curing of the liquid pre-gel with the scrim in situ, the hydrogel is thereby formed encapsulating the scrim material. Use of a scrim material in this way is found to be potentially helpful in enhancing the strength and ease of handling of the hydrogel component and/or the finished composite or dressing.

When present, the/each additional layer may be added in similar manner, namely either by prepolymerising the crosslinked hydrogel (optionally containing an embedded scrim, net or mesh in situ during the curing) and placing/laminating the latter down on to the growing laminar assembly with light pressure, or by placing/laminating the laminar assembly onto the polymerised hydrogel with light pressure; or, alternatively, a liquid pre-gel may be laid down onto the laminar assembly (optionally containing an embedded scrim, net or mesh) and the liquid pre-gel cured in situ. To those skilled in the art the performance and automation of such procedures will be well understood.

When any hydrogel material used in the composite or dressing contains water, the water may be present in any suitable amount. The typical range of water content is between 0 and about 95% by weight of the hydrogel. The hydrogel may conveniently be classified as “high water content” or “low water content”. The expression “high water content” refers particularly to hydrogel compositions comprising more than about 40% by weight of water, more particularly above about 50% by weight, and most preferably between about 60% and about 95% by weight. The expression “low water content” refers particularly to hydrogel compositions comprising up to about 40% by weight of water.

### The Net Structure

The net structure may, for example, be a porous fabric or mesh. It may, for example, be made from polymeric materials (natural, synthetic or a combination thereof) which may be selected from, but not limited to, polyethylene, polypropylene, nylon, polyester, poly(ethylene co vinyl acetate), cellulose, cellulose acetate and any combination thereof. The choice of material, thickness of material, pore size will be dictated by the need for the net structure to contact the absorbent hydrogel enabling the latter to directly contact the wound or other fluid source but also enabling the net structure to lower the adhesive properties absorbent hydrogel in the second layer.

The net structure has a thickness preferably less than about 0.3 mm, more preferably less than about 0.2 mm and more preferably less than about 0.1 mm, and most preferably less than about 0.08 mm but greater than about 0.02 mm. The apertures or pores of the net, which can be of any shape and size, should preferably each have an area greater than about 0.1 square mm, preferably greater than about 0.15, but less than about 2 square mm. Most preferably the net structure has a thickness of about 0.7 mm and a pore size between about 0.18 and about 0.3 square mm. An example of the preferred sizes is found in a polyethylene blend net SN09 from Smith and Nephew plc. (England)

Therapeutic agents may be impregnated/incorporated or attached or coated on to the net set like structure. For example exogenous growth factors and other biologically active molecules such as heparin andfibronectin may be used, Silver compounds, including but not limited to, silver nitrate, silver sulphadiazine, nano-crystalline silver and silver oxide may be incorporated to impart anti-microbial properties. Copper compounds including copper oxide may also be used as alternatives to silver. Other anti-microbial agents that can be used include, but are not limited to, iodine, chlorhexidine gluconate and polyhexamethylene biguanide.

### The Dressing—Physical Parameters

In the following description, the construction of a wound dressing for use on an exuding human skin wound is described. The adjustments required to make a hydrogel composite for other uses, or other articles embodying the hydrogel composite, will be well understood by those skilled in this art, and do not need to be described in detail.

The dressing may typically have a substantially uniform thickness. The dressing may typically have a thickness in the range of about 0.2 mm to about 10 mm. The dressing may suitably be in the form of a sheet having a mean basis weight of hydrogel in the range of about 0.1 kg/m² to about 4 kg/m².

The water activity, which is related to the osmolarity and the ionic strength of the precursor solution (as measured, for example, by a chilled mirror dewpoint meter, Aqualab T3) of the hydrogel wound dressings of the present invention, is preferably between 0.05 and 0.99, more preferably between, 0.2 and 0.99, and even more preferably between 0.3 and 0.98, for example between 0.6 and 0.89. The ionic strength of the precursor solution can therefore be used to optimise the hydrogel properties.

The dressing can have an area of between about 1 cm² and about 900 cm² and an overall thickness of between about 0.3 and about 5 mm.

The absorption capacity of the dressing will generally be between about 30% and about 20000%. More typically, the absorption capacity of the dressing will be between about 100% and about 10000%. The absorption capacity refers to the volume of water or other fluid absorbed by the hydrogel as a proportion of the starting volume of the hydrogel.

### Ingredients of the Hydrogel Composition

The preferred hydrogel composition of the present invention comprises a plasticised three-dimensional matrix of cross-linked polymer molecules, and has sufficient structural integrity to be self-supporting even at very high levels of internal water content, with sufficient flexibility to conform to the surface contours of the human skin. Our PCT Patent Application No. WO-00/45864, the disclosure of which is incorporated herein by reference, describes a method whereby the skin adhesion performance of the hydrogel can be predicted and thereby tailored to particular applications.

The hydrogel compositions with which the present invention is concerned generally comprise, in addition to the cross-linked polymeric network, an aqueous plasticising medium The materials and processing methods used are normally chosen to provide a suitable balance of adhesive and fluid handling properties for the desired application. For further details of the materials and methods of manufacture of individual component parts, please refer to the prior art documents acknowledged herein, as well as standard texts on hydrogels (e.g. “Hydrogels” in Kirk-Othmer Encyclopedia of Chemical Technology, 4^{th} Edition, vol. 7, pp. 783-907, John Wiley and Sons, N.Y., the contents of which are incorporated herein by reference).

### Monomers

The hydrogel component material may, for example, be a polymer of one or more ionic and/or non-ionic monomer.

Olefinically unsaturated sulphonic acid monomers are particularly suitable, and these include aliphatic or aromatic vinyl sulphonic acids such as vinylsulphonic acid, allylsulphonic acid, vinyltoluenesulphonic acid and styrene sulphonic acid; vinyl sulphobetaines such as SPDA (1-propanaminium N,N-dimethyl-N-[2-[(1-oxo-2-propenyl)oxy]-3-sulfo hydroxide, inner salt (available from Raschig); acrylic and methacrylic sulphonic acid such as sulphoethyl acrylate, sulphoethyl methacrylate, sulphopropyl acrylate, sulphopropyl methacrylate, 2-hydroxy-3-acryloxy propyl sulphonic acid, 2-hydroxy-3-methacryloxy propyl sulphonic acid and 2-acrylamido-2-methyl-propanesulphonic acid and salts (e.g. ammonium or alkali metal salts, such as sodium, potassium and lithium salts, or alkaline earth metal salts, such as calcium or magnesium) thereof.

The monomers may suitably be used in admixture with each other or with other monomers. In one particularly useful embodiment of the invention, a monomer which has a first countercation associated with it may be used in admixture with one or more monomer which has/have one or more second/further countercation(s) associated with it/them. The monomers in their anionic form (i.e. disregarding the counter-cation) may be the same or different. In this way, the proportions of different cations (e.g. alkali metal ions such as sodium or potassium, or primary, secondary, tertiary or quaternary ammonium ions) can be finely controlled in the resultant polymer (homopolymer or copolymer). The particular weight ratios of one monomer to the or each other monomer, and/or the respective countercations, can be selected within wide limits by those skilled in the art, depending on the desired properties of the resultant hydrogel polymer.

Further examples of suitable monomers for use in the present invention include: a polyalkylene glycol acrylate or a substituted derivative thereof; a polyalkylene glycol methacrylate or a substituted derivative thereof; acrylic acid and salts thereof (e.g. alkali metal salts such as sodium, potassium and lithium salts); 2-acrylamido-2-methyl-propanesulphonic acid and salts thereof (e.g. ammonium or alkali metal salts, such as sodium, potassium and lithium salts, or alkaline earth metal salts, such as calcium or magnesium); acrylic acid (3-sulphopropyl) ester or a substituted derivative thereof or a salt thereof (e.g. an alkali metal salt such as sodium, potassium or lithium salt); diacetone acrylamide (N-1,1-dimethyl-3-oxobutyl-acrylamide); a vinyl lactam (e.g. N-vinyl pyrrolidone or a substituted derivative thereof); an optionally substituted N-alkylated acrylamide such as hydroxyethyl acrylamide; and an optionally substituted N,N-dialkylated acrylamide; and/or N-acryloyl morpholine or a substituted derivative thereof.

Particularly preferred monomers include: the sodium salt of 2-acrylamido-2-methylpropane sulphonic acid, commonly known as NaAMPS, which is available commercially at present from Lubrizol as either a 50% aqueous solution (reference code LZ2405) or a 58% aqueous solution (reference code LZ2405A); the potassium salt of 2-acrylamido-2-methylpropane sulphonic acid (Potassium AMPS), which is available commercially at present from Lubrizol; the ammonium salt of 2-acrylamido-2-methylpropane sulphonic acid (Ammonium AMPS), which is available commercially at present from Lubrizol; acrylic acid (3-sulphopropyl) ester potassium salt, commonly known as SPA or SPAK (SPA or SPAK is available commercially in the form of a pure solid from Raschig); acrylic acid (3-sulphopropyl) ester sodium salt, commonly known as SPANa (SPANa is available in the form of a pure solid from Raschig); and SPDA. Acrylic acid (BASF) may be used as supplied or in partial or complete salt form where the salt counterion is an alkali metal (e.g. sodium or potassium), alkaline earth metal (e.g. calcium) or ammonium. Mixtures of any two or more of the above monomers may be used. When a mixture of the monomers is used, it may, for example, be a mixture of NaAMPS and SPAK, a mixture of NaAMPS and SPANa, a mixture of NaAMPS and Potassium AMPS, a mixture of NaAMPS and Ammonium AMPS, or a mixture of NaAMPS and acrylic acid. The relative amounts of the monomers in a mixture will be dictated by the desired ratio of counterions (e.g. potassium, sodium and ammonium) in the hydrogel, as well as the required properties of the copolymer, and may be selected easily by those skilled in the art, if necessary with routine testing of the copolymers prepared.

The above monomers and monomer types may optionally include substituent groups. Optional substituents of the monomers used to prepare the hydrogels used in the present invention may preferably to selected from substituents which are known in the art or are reasonably expected to provide polymerisable monomers which form hydrogel polymers having the properties necessary for the present invention. Suitable substituents include, for example, lower (C1 to C6) alkyl, hydroxy, halo and amino groups.

### Cross-linking Agents

Conventional cross-linking agents are suitably used to provide the necessary mechanical stability and to control the adhesive properties of the hydrogel. The amount of cross-linking agent required to produce a crosslinked absorbent hydrogel for direct contact with the wound, which is resistant break up on the absorption of fluid to is from 0.04 to about 0.3%, more preferably between about 0.08 and about 0.2%, by weight of the total polymerisation reaction mixture. Typical cross-linkers include tripropylene glycol diacrylate, ethylene glycol dimethacrylate, triacrylate, polyethylene glycol diacrylate (polyethylene glycol (PEG) molecular weight between about 100 and about 4000, for example PEG400 or PEG600), and methylene bis acrylamide. When two ore more hydrogels layers are employed in the absorbent hydrogel second layer of the wound dressing the preferred amount of cross-linking agent the amount of crosslinking agent used can be the same but is preferably less than that used for the wound contacting hydrogel.

### Organic Plasticisers

The one or more organic plasticiser, when present, may suitably comprise any of the following either alone or in combination: at least one polyhydric alcohol (such as bot limited to honey, glycerol, polyethylene glycol, or sorbitol), at least one ester derived therefrom, at least one polymeric alcohol (such as polyethylene oxide) and/or at least one mono- or poly-alkylated derivative of a polyhydric or polymeric alcohol (such as alkylated polyethylene glycol). Glycerol is the preferred plasticiser. An alternative preferred plasticiser is the ester derived from boric acid and glycerol. When present, the organic plasticiser may comprise up to about 60% by weight of the hydrogel composition.

### Surfactants

Any compatible surfactant may optionally be used as an additional ingredient of the hydrogel composition. Surfactants can lower the surface tension of the mixture before polymerisation and thus aid processing. Non-ionic, anionic and cationic surfactants are preferred. The surfactant ideally comprises any of the surfactants listed below either alone or in combination with each other and/or with other surfactants. The total amount of surfactant, if present, is suitably up to about 10% by weight of the hydrogel composition, preferably from about 0.05% to about 4% by weight.

### Other Additives

The hydrogel in the composite of the present invention may include one or more additional ingredients, which may be added to the pre-polymerisation mixture or the polymerised product, at the choice of the skilled worker. Such additional ingredients are selected from additives known in the art, including, for example, water, organic plasticisers, surfactants, polymeric material (hydrophobic or hydrophilic in nature, including proteins, enzymes, naturally occurring polymers and gums), synthetic polymers with and without pendant carboxylic acids, electrolytes, osmolites, pH regulators, colorants, chloride sources, bioactive compounds and mixtures thereof. The polymers can be natural polymers (e.g. xanthan gum), synthetic polymers (e.g. polyoxypropylene-polyoxyethylene block copolymer or poly-(methyl vinyl ether alt maleic anhydride)), or any combination thereof. By “bioactive compounds” we mean any compound or mixture included within the hydrogel for some effect it has on living systems, whether the living system be bacteria or other microorganisms or higher animals such as the patient. Bioactive compounds that may be mentioned include, for example, pharmaceutically active compounds, antimicrobial agents, antiseptic agents, antibiotics and any combination thereof. Antimicrobial agents may, for example, include: sources of oxygen and/or iodine (e.g. hydrogen peroxide or a source thereof and/or an iodide salt such as potassium iodide) (see, for example Bioxzyme™ technology, for example in The Sunday Telegraph (UK) 26 Jan. 2003 or the discussion of the Oxyzyme™ system at www.wounds-uk.com/posterabstracts2003.pdf); honey (e.g. active Manuka honey); antimicrobial metals, metal ions and salts, such as, for example, silver-containing antimicrobial agents (e.g. colloidal silver, silver oxide, silver nitrate, silver thiosulphate, silver sulphadiazine, or any combination thereof), hyperchlorous acid; or any combination thereof and copper based agents (e.g. salts complexes and/or dispersions)

In the Bioxzyme system, a dressing comprises two hydrogels. One contains glucose based antibacterial compounds and the other contains enzymes that convert the glucose into hydrogen peroxide. When these are exposed to air and contacted together at a wound site, the enzyme-containing gel being adjacent the skin and the glucose-containing gel overlying the enzyme-containing gel, a low level steady flow of hydrogen peroxide is produced, which inhibits anaerobic bacteria. This antibacterial effect can be enhanced by the inclusion of a very low level of iodide (less than about 0.04%) in the hydrogel. The hydrogen peroxide and the iodide react to produce iodine, a potent antimicrobial agent.

Hydrogels incorporating antimicrobial agents may, for example, be active against such organisms as Staphylococcus aureus and Pseudomonas aeruginosa.

Agents for stimulating the healing of wounds and/or for restricting or preventing scarring may be incorporated into the hydrogel. Examples of such agents include growth factors such as TGF (transforming growth factor), PDGF (platelet derived growth factor), KGF (keratinocyte growth factor, e.g. KGF-1 or KGF-2), VEGF (vascular endothelial growth factor), IGF (insulin growth factor, optionally in association with one or more of IGF binding protein and vitronectin), e.g. from GroPep Ltd, Australia or Procyte, USA (see, e.g. WO-A-96/02270, the contents of which are incorporated herein by reference); cell nutrients (see, e.g., WO-A-93/04691, the contents of which are incorporated herein by reference); glucose (see, e.g., WO-A-93/10795, the contents of which are incorporated herein by reference); an anabolic hormone or hormone mixture such as insulin, triiodothyronine, thyroxine or any combination thereof (see, e.g., WO-A-93/04691, the contents of which are incorporated herein by reference); or any combination thereof.

Additional polymer(s), typically rheology modifying polymer(s), may be incorporated into the polymerisation reaction mixture at levels typically up to about 10% by weight of total polymerisation reaction mixture, e.g. from about 0.2% to about 10% by weight. Such polymer(s) may include polyacrylamide, poly-NaAMPS, polyethylene glycol (PEG), polyvinylpyrrolidone (PVP) or carboxymethyl cellulose.

Additional osmolite(s) may be included to modify the osmolarity of the hydrogel. Osmolites may be ionic (e.g. electrolytes, for example salts which are readily soluble in the aqueous phase of the hydrogel to increase the ionic strength of selected cations or anions and hence the osmolarity of the hydrogel). By selecting the ions present in an ionic osmolite, and particularly by selecting the cation so as to correspond or not with cationic counterions in the monomer(s) of the hydrogel, the ionic strength of certain anions (e.g. chloride) can be varied with fine control, without substantially changing the ionic strength of cations already present in very large amounts as counterions of the monomer(s).

Osmolites may be organic (non-ionic), for example organic molecules which dissolve in or intimately mix with the aqueous phase of the hydrogel to increase the osmolarity of the hydrogel deriving from non-ionic species in the aqueous phase. Such organic osmolites include, for example, water-soluble sugars (e.g. glucose and other monosaccharides), polyhydric alcohols (e.g. glycerol and other polyhydroxylated alkanols).

Additive ingredients may serve more than one purpose. For example, glycerol may serve as an organic plasticiser and an osmolite.

The hydrogel may comprise one or more complexing or chelating agents, which may include, but are not limited to, organic poly-carboxylic acids, and includes, but is not limited to, agents that can form complexes with or chelate to one or more metal ions. The complexing agent may be selected from di-, tri- and tetra- carboxylic acids. Preferably, the one or more complexing or chelating agents contain a moiety in which two carboxylic acid groups (CO₂H) or salts thereof are separated by three or four covalent bonds (e.g. three bonds in malic acid: (HO₂C)—CH₂—CH(OH)—(CO₂H); four bonds in EDTA: (HO₂C)—CH₂—NR—CH₂—(CO₂H), in which R is the remaining part of the molecule). The complexing or chelating agents may comprise one or more molecules containing one or more primary, secondary or tertiary nitrogens within their structure. The complexing or chelating agents may include, but are not limited to, EDTA, citric acid, maleic acid, malic acid, and their salts (which include, but are not limited to, sodium and potassium salts). These agents have been found to be effective in controlling any ion exchange that may be associated with the hydrogel composition.

The hydrogel used in the present invention preferably consists essentially of a cross-linked hydrophilic polymer of a hydrophilic monomer and optionally one or more comonomer, together with water and/or one or more organic plasticiser, and optionally together with one or more additives selected from surfactants, polymers, pH regulators, electrolytes, osmolites, chloride sources, bioactive compounds and mixtures thereof, with less than about 40%, for example less than about 10%, by weight of other additives.

### Preparative Method for the Hydrogels—General

The processes for the preparation of hydrogels generally comprise polymerising a polymerisable mixture comprising at least one hydrophilic monomer.

In addition to the at least one hydrophilic monomer, a curing system should be present in the polymerisable mixture. The curing system typically includes at least one cross-linking agent and at least one suitable polymerisation initiator.

The polymerisation is preferably a free radical polymerisation of a fluid polymerisable mixture comprising

(1) a free radically polymerisable hydrophilic monomer, optionally together with at least one free radically polymerisable comonomer; and

(2) one or more cross-linking agent comprising a multifunctional unsaturated free radically polymerisable compound;

the polymerisation being conducted in the presence or absence of a plasticiser, with the proviso that when the polymerisation is conducted in the absence of a plasticiser, a plasticiser is added to the polymer product of the polymerisation.

The polymerisable mixture (pre-gel) preferably includes the monomer(s) at a total monomer level of from about 5% to about 70% by weight of the total mixture, more particularly from about 10% to about 60% by weight, most preferably from about 15% to about 50% by weight.

When the polymerisation is conducted in the presence of a plasticiser, one or more different plasticiser and/or more of the same plasticiser may, if desired, be added to the polymer product of the polymerisation.

### Uses

The hydrogel composites described herein may be used in a range of skin contact or covering applications where the composition is brought into contact either with skin or with an intermediary member which interfaces between the composite and the skin.

The composite may be unsupported or may be supported on a part of a larger article for some specific use, e.g. a backing structure. Applications include patches, tapes, bandages, devices and dressings of general utility or for specific uses, including without limitation biomedical, skin care, personal and body care, palliative and veterinary uses such as, for example, skin electrodes for diagnostic (e.g. ECG), stimulation (e.g. TENS), therapeutic (e.g. defibrillation) or electrosurgical (e.g. electrocauterisation) use; dressings and reservoirs for assisting wound and burn healing, wound and burn management, skin cooling, skin moisturizing, skin warming, aroma release or delivery, decongestant release or delivery, pharmaceutical and drug release or delivery, perfume release or delivery, fragrance release or delivery, scent release or delivery, and other skin contacting devices such as absorbent pads or patches for absorbing body fluids (e.g. lactation pads for nursing mothers), hairpiece adhesives and clothing adhesives; and adhesive flanges and tabs for fecal collection receptacles, ostomy devices and other incontinence devices.

For further details of suitable hydrogel material for use in the present invention, and its preparation, please refer to the following publications: PCT Patent Applications Nos. WO-97/24149, WO-97/34947, WO-00/06214, WO-00/06215, WO-00/07638, WO-00/46319, WO-00/65143 and WO-01/96422, the disclosures of which are incorporated herein by reference.

### BRIEF DESCRIPTION OF THE DRAWINGS

The invention will now be described further, without limitation and purely by way of example, with reference to the accompanying drawings, in which:

FIG. 1 illustrates in schematic transverse cross-section the internal layer structure of an absorbent hydrogel wound dressing according to the present invention; and

FIG. 2 illustrates in schematic transverse cross-section the internal layer structure of an alternative absorbent hydrogel wound dressing according to the present invention.

### DETAILED DESCRIPTION OF THE DRAWINGS

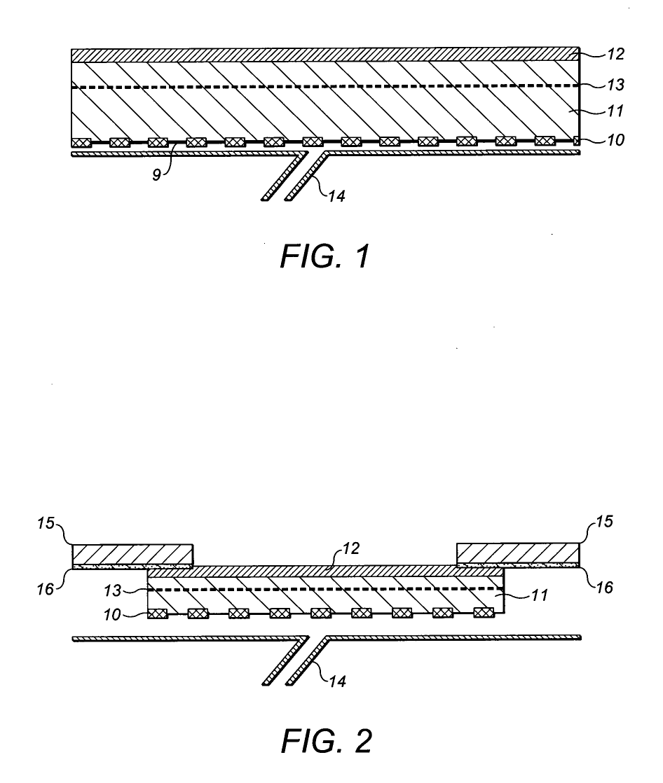

Hydrogel wound dressings of the present invention are illustrated in FIGS. 1 and 2.

FIG. 1 shows the laminar structure comprising three main layers 10, 11 and 12. Layer 10 is the wound contacting net structure adhered to the surface of the absorbent hydrogel second layer 11 such that the hydrogel 11 can directly contact the wound through the apertures 9 of the net 10 (not shown). Layer 11 comprises the main body of the absorbent hydrogel. A reinforcing scrim 13 is also shown, embedded within hydrogel layer 11. On the wound contacting face is shown a release liner 14, for removal prior to application to the wound.

Layer 12 comprises a overlying, non-hydrogel, polymeric backing layer.

In FIG. 2 the hydrogel wound dressing is as in FIG. 1, but with the addition of a fourth layer 15, which in this case extends beyond the border of the structure 10/11/12 containing the absorbent hydrogel, and comprises a fenestrated film with a skin contacting adhesive 16 on the underneath surface.

### EXAMPLES

The invention will be further described with reference to the following Examples, which do not limit the scope of the invention.

In these Examples, each of the pre-gel formulations was cured as 0.3 to 2.6 kg per square metre coat weight by a medium pressure mercury arc lamp located within a bench top UV curing machine (NUVA-Solo-30, GEW, UK). at a conveyer speed of 7 m/minute.

### Example 1

A glass petri dish (circa 9 cm diameter) was lined with a polyurethane film (Inspire 2301, Intelicoat UK). Circa 1.5 to 2 g of the following pre-gel formulation was evenly spread across the petri dish. A polyester scrim (HDK D1451, circa 9 cm diameter) was placed on top of the pre-gel coated polyurethane. About 4 to 5 g of the following pre-gel formulation were evenly spread into the lined petri dish on top of the polyester scrim. The hydrogel was then cured.

Pre-gel: 67 parts by weight of 58% aqueous solution of the sodium salt of acrylamidomethyl-propanesulphonic acid (NaAMPS, LZ2405 Lubrizol), 0.5 parts acrylic acid (3-sulphopropyl) ester potassium salt, commonly known as SPA or SPAK (SPA or SPAK is available commercially in the form of a pure solid from Raschig), 30 parts glycerol. These ingredients are mix A. Into 100 g of mix A, 0.11 g of a 2:10 (by weight) mixture of Daracure 1173 photoinitiator (Ciba Speciality Chemicals) and IRR280 cross-linker (PEG400 diacrylate, UCB Chemicals) is added and stirred for one hour. The hydrogel was then cured. This is hydrogel structure A.

On top of the cured hydrogel structure A described above, a further 4 g of the following pre-gel formulation were evenly spread and then cured to form hydrogel structure B.

Pre-gel: 67 parts by weight of 58% aqueous solution of the sodium salt of acrylamidomethyl-propanesulphonic acid (NaAMPS, LZ2405 Lubrizol), 0.5 parts acrylic acid (3-sulphopropyl) ester potassium salt, commonly known as SPA or SPAK (SPA or SPAK is available commercially in the form of a pure solid from Raschig), 30 parts glycerol. These ingredients are mix A. Into 100 g of mix A, 0.15 g of a 2 to 10 (by weight) mixture of Daracure 1173 photoinitiator (Ciba Speciality Chemicals) and IRR280 cross-linker (PEG400 diacrylate, UCB Chemicals) was added and stirred for one hour. The hydrogel was then cured. This is hydrogel structure B.

A circular piece (circa 9 cm diameter) of net (SN09, Smith and Nephew) was then laid on top of hydrogel structure B.

A 9 cm diameter circular piece of siliconised polyethylene was laminated to the wound contacting face of the wound dressing.

The assembled hydrogel wound dressing corresponds to that shown in FIG. 1.

### Example 2

A glass petri dish (circa 9 cm diameter) was lined with a polyurethane film (Inspire 2301, Intelicoat UK). Circa 1.5 to 2 g of the following pre-gel formulation was evenly spread across the petri dish. A polyester scrim (HDK D1451, circa 9 cm diameter) was placed on top of the pre-gel coated polyurethane. About 4 to 5 g of the following pre-gel formulation were evenly spread into the lined petri dish on top of the polyester scrim. The hydrogel was then cured.

Pre-gel: 67 parts by weight of 58% aqueous solution of the sodium salt of acrylamidomethyl-propanesulphonic acid (NaAMPS, LZ2405 Lubrizol), 0.5 parts acrylic acid (3-sulphopropyl) ester potassium salt, commonly known as SPA or SPAK (SPA or SPAK is available commercially in the form of a pure solid from Raschig), 30 parts glycerol. These ingredients are mix A. Into 100 g of mix A, 0.11 g of a 2 to 10 (by weight) mixture of Daracure 1173 photoinitiator (Ciba Speciality Chemicals) and IRR280 cross-linker (PEG400 diacrylate, UCB Chemicals) is added and stirred for one hour. The hydrogel was then cured. This is hydrogel structure A.

On top of the cured hydrogel structure described above a further 4 g of the following pre-gel formulation were evenly spread and then cured to form hydrogel structure B.

Pre-gel: 67 parts by weight of 58% aqueous solution of the sodium salt of acrylamidomethyl-propanesulphonic acid (NaAMPS, LZ2405 Lubrizol), 0.5 parts acrylic acid (3-sulphopropyl) ester potassium salt, commonly known as SPA or SPAK (SPA or SPAK is available commercially in the form of a pure solid from Raschig), 30 parts glycerol. These ingredients are mix A. Into 100 g of mix A, 0.15 g of a 2 to 10 (by weight) mixture of Daracure 1173 photoinitiator (Ciba Speciality Chemicals) and IRR280 cross-linker (PEG400 diacrylate, UCB Chemicals) was added and stirred for one hour. The hydrogel was then cured. This is hydrogel structure B.

Hydrogel structure B was then laid on top of a circular piece (circa 9 cm diameter) of net (SN09, Smith and Nephew).

The assembled hydrogel wound dressing corresponds to that shown schematically in FIG. 1.

### Example 3

A circular piece 12 cm in diameter of an adhesive coated polyurethane, Inspire 2317, Intelicoat UK was laminated as an overlayer to the polyurethane film of Example 1.

### Example 4

A circular piece 12 cm in diameter of an adhesive coated polyurethane, Inspire 2317, Intelicoat UK, with a centrally placed hole 8 cm in diameter was laminated as a fenestrated overlayer to the polyurethane film of example 1 as shown schematically in FIG. 2.

### Example 5

To test for the resistance of the absorbent hydrogel in the wound dressing of the present invention to break-up on absorbing fluid, the following test was conducted using pure water or isotonic solutions.

Apparatus:
Gel
   circle template 5 cm diameter
Scissors
Balance
Paddington cups (Surgical Materials Testing Laboratory, Cardiff, UK)
Purified water
Normal Saline solution (0.88 to 0.92 g NaCl and purified water to 100 g)
Calcium Saline solution (0.81 to 0.85 g NaCl and 0.027 to 0.029 g CaCl₂ and purified water to 100 g; analytical grade anhydrous salts were used to prepare the isotonic solutions)
Torque controlled screwdriver
Tray
Oven at 40° C., 55% RH

Method:
1. Lay the sample down on a flat surface, and draw a circle using the template. Cut the circle out. Cut two circles for each sample to be analysed.
2. Take a Paddington cup, place on the balance and record the mass. Fill the Paddington cup with 20 g of test fluid (pure water, Normal Saline or Calcium Saline—Note: Calcium Saline is a synthetic wound fluid also known as ‘Solution A’ in BP (1995) and BS EN 13726-1:2002), and record the mass. Record which fluid is used for each test.
3. Remove the lid from the Paddington cup.
4. Remove the liners from one side of the sample, and carefully stick the sample down, ensuring that there are no wrinkles, that the central hole is covered with gel, but that the screw holes are not.
5. Remove the liners from the upper surface of the gel. Put the lid back on and tighten the screws using the screwdriver until a torque of 40 cN·m is reached.
6. Record the mass of the Paddington cup.
7. Repeat for all samples.
8. When all the Paddington cups have been prepared, place the samples on a tray, in an inverted position such that the fluid directly contacts the dressing, and place the tray in the oven.
9. Record the time at which the samples are placed in the oven.
10. After 4 hours remove the samples, and measure and record their masses.

Calculations:

$$MVTR ( g \text{/} {m}^{2} \text{/} 24 hrs ) = \frac{\begin{matrix}( Final mass of cup + fluid + sample ) - \\ ( Initial weight of cup + fluid + sample )\end{matrix}}{0.001}$$

Hypothetical example of the calculation:

| | | Mass | | | | Ab- | |
|---|---|---|---|---|---|---|---|
| | | cup | +fluid | +sample | +4 hrs | sorption | MVTR |
| Sample | Cup | g | g | g | g | g | g/m²/24 h |
| Example | A | 66.65 | 86.65 | 89.37 | 88.01 | 1.36 | 1360 |

Using the above method and Calcium Saline as the test fluid, the wound dressings of Examples 1 and 2 were found to exhibit minimal gel break. They had absorbed about 4.5 g of fluid. The data and calculation are shown in the table below:

| | | Mass | | +sam- | | Ab- | |
|---|---|---|---|---|---|---|---|
| | | cup | +fluid | ple | +4 hrs | sorption | MVTR |
| Sample | Cup | g | g | g | g | g | g/m²/24 h |
| Example | A | 166.61 | 186.61 | 189.43 | 188.49 | 4.49 | 940 |
| 1 | | | | | | | |
| Example | B | 166.59 | 186.59 | 189.43 | 188.52 | 4.51 | 910 |
| 2 | | | | | | | |

### INDUSTRIAL APPLICABILITY

The present invention makes available absorbent hydrogel composites and wound dressings with useful capacity to absorb potentially large quantities of liquids at an acceptable speed for many uses with greatly reduced or minimised break-up of the hydrogel. In particular, the hydrogel wound dressing enables the hydrogel to directly contact the wound which allows the intrinsic wound stimulating and pain relieving properties of the hydrogel to be effectively utilised. Moreover, the hydrogels can be made conveniently and efficiently.

The present invention has been broadly described without limitation. Variations and modifications as will be readily apparent to those skilled in the art are intended to be covered by the present application and resultant patents.

1. An absorbent hydrogel composite for application to a fluid-emitting surface, the composite having a laminar structure comprising first and second layers, the first layer being a fluid-emitting-surface-contacting layer comprising a porous net structure having a fluid-emitting-surface-contacting face and an outwardly directed face, the second layer comprising a low-crosslinked absorbent hydrogel disposed over the outwardly directed face of the first layer and arranged so that in use it is in fluid flow communication with the fluid-emitting surface through at least some apertures of the net structure.

2. The absorbent hydrogel composite according to claim 1, wherein the second layer contacts the fluid-emitting surface through at least some of the apertures of the first layer.

3. The absorbent hydrogel composite according to claim 1, wherein the porous net structure has a thickness of 0.3 mm or less.

4. The absorbent hydrogel composite according to claim 1, wherein at least some of the apertures have an area of 0.1 square mm or more.

5. The absorbent hydrogel composite according to claim 3, wherein the porous net structure has a thickness between 0.02 mm to 0.1 mm and at least some of the apertures have an area in the range of 0.1 square mm to 2 square mm.

6. The absorbent hydrogel composite of claim 1, wherein the absorbent hydrogel composite has an adhesion level of 4N/25 mm in a 180 degree peel test at a speed of 200 mm/minute.

7. The absorbent hydrogel composite of claim 1, wherein the low-crosslinked absorbent hydrogel has been formed from a polymerisation mixture comprising a cross-linking agent in an amount of 0.04 to 0.3% by weight of the polymerisation mixture.

8. The absorbent hydrogel composite of claim 1 further comprising a third layer in direct contact with and overlying the outward face of the second layer, the third layer have one or more apertures therethrough.

9. The absorbent hydrogel composite according to claim 8, wherein the third layer has a single aperture therein.

10. The absorbent hydrogel composite according to claim 8 or 9, wherein the one or more apertures or single aperture have/has a total area of 10 to 95% of the area of the second layer.

11. The absorbent hydrogel composite according to claim 7, wherein the third layer extends beyond the margins of the first and second layers and has an adhesive on its underside for adhesion of the third layer to skin surrounding a wound.

12. The absorbent hydrogel composite of claim 1, wherein the moisture vapour transmission rate (MVTR) of the absorbent hydrogel composite is from 150 to about 2500 g/m2/24hours, as measured at 40° C., and 55% relative humidity.

13. The absorbent hydrogel composite of claim 1, wherein a scrim material is integral with the low-crosslinked absorbent hydrogel of the second layer.

14. (canceled)

15. (canceled)

16. A wound dressing comprising an absorbent hydrogel composite having a laminar structure comprising first and second layers, the first layer being a fluid-emitting-surface-contacting layer comprising a porous net structure having a fluid-emitting-surface-contacting face and an outwardly directed face, the second layer comprising a low-crosslinked absorbent hydrogel disposed over the outwardly directed face of the first layer and arranged so that in use it is in fluid flow communication with the fluid-emitting surface through at least some apertures of the net structure.

17. The wound dressing according to claim 16, having a laminar structure comprising first and second layers, the first layer being a wound contacting layer comprising a porous net structure having a wound contacting face and an outwardly directed face, the second layer comprising a low-crosslinked absorbent hydrogel disposed over the outwardly directed face of the first layer and arranged so that in use it is in fluid flow communication with the wound through at least some apertures of the net structure.

18. The wound dressing according to claim 17, wherein the second layer contacts the fluid-emitting surface through at least some of the apertures.

19. (canceled)

20. The wound dressing of claim 17, further comprising a third layer in direct contact with and overlying the outward face of the second layer, the third layer having one more apertures therethrough.

21. A method for treating a chronic ulcerous skin lesion, the method comprising: contacting a chronic ulcerous skin lesion with an absorbent hydrogel composite of claim 1 or a wound dressing having a laminar structure comprising first and second layers, the first layer being a wound contacting layer comprising a porous net structure having a wound contacting face and an outwardly directed face, the second layer comprising a low-crosslinked absorbent hydrogel disposed over the outwardly directed face of the first layer and arranged so that it is in fluid flow communication with the wound through at least some apertures of the net structure.

22. A method for treating a wound, the method comprising: contacting the wound with an absorbent hydrogel composite having a laminar structure comprising first and second layers, the first layer being a fluid-emitting-surface-contacting layer comprising a porous net structure having a fluid-emitting-surface-contacting face and an outwardly directed face, the second layer comprising a low-crosslinked absorbent hydrogel disposed over the outwardly directed face of the first layer and arranged so that in use it is in fluid flow communication with the fluid-emitting surface through at least some apertures of the net structure.
